(19) United States
(12) Patent Application Publication
(10) Pub. No.: US 2011/0253994 A1
(43) Pub. Date: Oct. 20, 2011

(54) COMPOUND FOR ORGANIC OPTOELECTRONIC DEVICE, ORGANIC LIGHT EMITTING DIODE INCLUDING THE SAME AND DISPLAY DEVICE INCLUDING THE SAME

(76) Inventors: Hyung-Sun Kim, Uiwang-si (KR); Ho-Jae Lee, Uiwang-si (KR); Sung-Hyun Jung, Uiwang-si (KR); Young-Sung Park, Uiwang-si (KR); Eun-Sun Yu, Uiwang-si (KR); Mi-Young Chae, Uiwang-si (KR); Young-Hoon Kim, Uiwang-si (KR); Ja-Hyun Kim, Bryeong-city (KR)

(21) Appl. No.: 13/171,969

(22) Filed: Jun. 29, 2011

Related U.S. Application Data
(63) Continuation of application No. PCT/KR2009/007400, filed on Dec. 10, 2009.

(30) Foreign Application Priority Data
Dec. 29, 2008 (KR) 10-2008-0135689

(51) Int. Cl. H01L 51/30; C07D 235/18; C07D 413/04; C07D 271/107

(52) U.S. Cl. 257/40; 548/143; 548/310.7; 546/269.4; 257/E51.026

(57) ABSTRACT

A compound for an organic optoelectronic device, an organic optoelectronic device including the same, and a display device including the same, the compound for an organic optoelectronic device being represented by the following Chemical Formula 1:

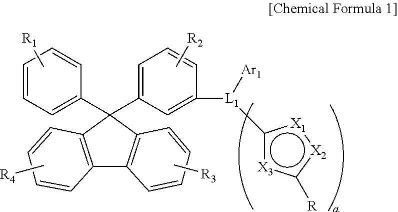

### CROSS-REFERENCE TO RELATED APPLICATION

This application is a continuation of pending International Application No. PCT/KR2009/007400, entitled “Novel Compounds for an Organic Photoelectric Device, and Organic Photoelectric Device Comprising Same,” which was filed on Dec. 10, 2009, the entire contents of which are hereby incorporated by reference.

### FIELD

Embodiments relate to a compound for an organic optoelectronic device, an organic light emitting diode including the same, and a display device including the same.

### DESCRIPTION OF THE RELATED ART

An optoelectronic device is, in a broad sense, a device for transforming photo-energy to electrical energy, and conversely, for transforming electrical energy to photo-energy. For example, among these photoelectric devices, an organic light emitting diode has recently drawn attention due to the increase in demand for flat panel displays.

The organic light emitting diode has a structure in which a functional organic material layer is interposed between an anode and a cathode. The organic thin layer may include an emission layer, a hole injection layer (HIL), a hole transport layer (HTL), an electron transport layer (ETL), an electron injection layer (EIL), and the like, and may further include an electron blocking layer or a hole blocking layer in terms of light emitting properties of the emission layer.

An organic light emitting diode may include a laminated structure of an organic thin layer including a hole transport layer (HTL) of a low molecular aromatic diamine derivative and an electron transport emission layer of tris(8-hydroxy-quinolate) aluminum (Alq₃). However, electrical characteristics and life-span may still be improved.

In an organic light emitting diode, holes are injected from an anode and electrons are injected from a cathode. The holes and electrons are transported to opposite electrodes and recombined therewith and form excitons having high energy. The provided light emitting excitons emit light having a predetermined wavelength by transiting to the ground state. The light emission may be classified as a fluorescent material (including singlet excitons) and a phosphorescent material (including triplet excitons) according to light emitting mechanism. A phosphorescent light emitting material may be used for a light emitting material of an organic light emitting diode in addition to the fluorescent light emitting material

Such a phosphorescent material emits lights by transiting the electrons from a ground state to an exited state, non-radiance transiting of a singlet exciton to a triplet exciton through intersystem crossing, and transiting a triplet exciton to a ground state to emit light. When the triplet exciton is transited, it cannot directly transit to the ground state. Therefore, the electron spin is flipped, and then it is transited to the ground state. Therefore, a phosphorescent light emitting material has longer light emitting duration than a fluorescent light emitting material. The phosphorescent light emitting material may show about four times higher luminous efficiency than a fluorescent light emitting material.

### SUMMARY

Embodiments are directed to a compound for an organic optoelectronic device, an organic light emitting diode including the same, and a display device including the same.

The embodiments may be realized by providing a compound for an organic optoelectronic device, the compound being represented by the following Chemical Formula 1:

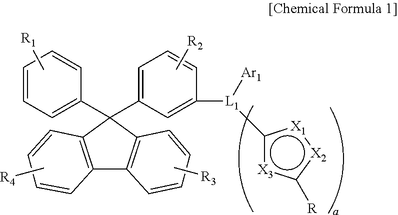

wherein, in Chemical Formula 1, Ar₁ is hydrogen or a substituted or unsubstituted C18 to C40 fluorenyl group, L₁ is selected from the group of a substituted or unsubstituted C1 to C30 alkylene group, a substituted or unsubstituted C6 to C30 arylene group, a substituted or unsubstituted C1 to C30 heteroarylene group, and N, X₁ to X₃ are each independently selected from the group of N, NR′₁, O, S, and CR′₂, provided that at least two of X₁ to X₃ are selected from the group of N, O, and S, R, R′₁, and to R′₂ are each independently selected from the group of a substituted or unsubstituted C1 to C30 alkyl group, a substituted or unsubstituted C1 to C30 alkylene group, a substituted or unsubstituted C2 to C30 alkenyl group, a substituted or unsubstituted C2 to C30 alkenylene group, a substituted or unsubstituted C6 to C30 aryl group, and a substituted or unsubstituted C1 to C30 heteroaryl group, and optionally adjacent two of R, R′₁, and to R′₂ form a fused ring, a is an integer of 1 to 4, and R₁ to R₄ are each independently hydrogen or a substituted or unsubstituted C1 to C10 alkyl group.

The compound represented by Chemical Formula 1 may include a compound represented by the following Chemical Formula 2:

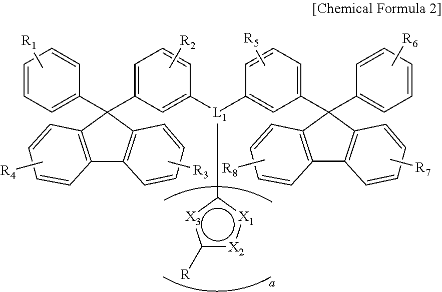

wherein in Chemical Formula 2, L₁ is selected from the group of a substituted or unsubstituted C1 to C30 alkylene group, a substituted or unsubstituted C6 to C30 arylene group, a substituted or unsubstituted C1 to C30 heteroarylene group, and N, X₁ to X₃ are each independently selected from the group of N, NR′₁, O, S, and CR′₂, provided that at least two of X₁ to X₃ are selected from the group of N, O, and S, R, R′₁, and to R′₂ are each independently selected from the group of a substituted or unsubstituted C1 to C30 alkyl group, a substituted or unsubstituted C1 to C30 alkylene group, a substituted or unsubstituted C2 to C30 alkenyl group, a substituted or unsubstituted C2 to C30 alkenylene group, a substituted or unsubstituted C6 to C30 aryl group, and a substituted or unsubstituted C1 to C30 heteroaryl group, and optionally adjacent two of R, R′₁, and to R′₂ form a fused ring, a is an integer of 1 to 4, and R₁ to R₈ are each independently hydrogen or a substituted or unsubstituted C1 to C10 alkyl group.

The compound represented by Chemical Formula 1 may include a compound represented by one of the following Chemical Formulae 3 to 26:

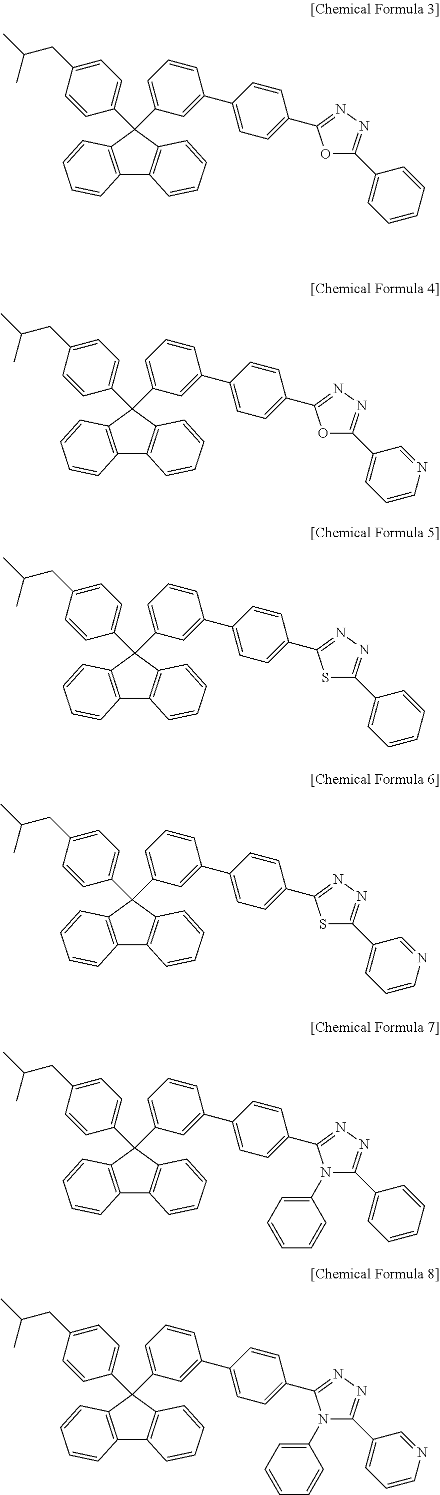

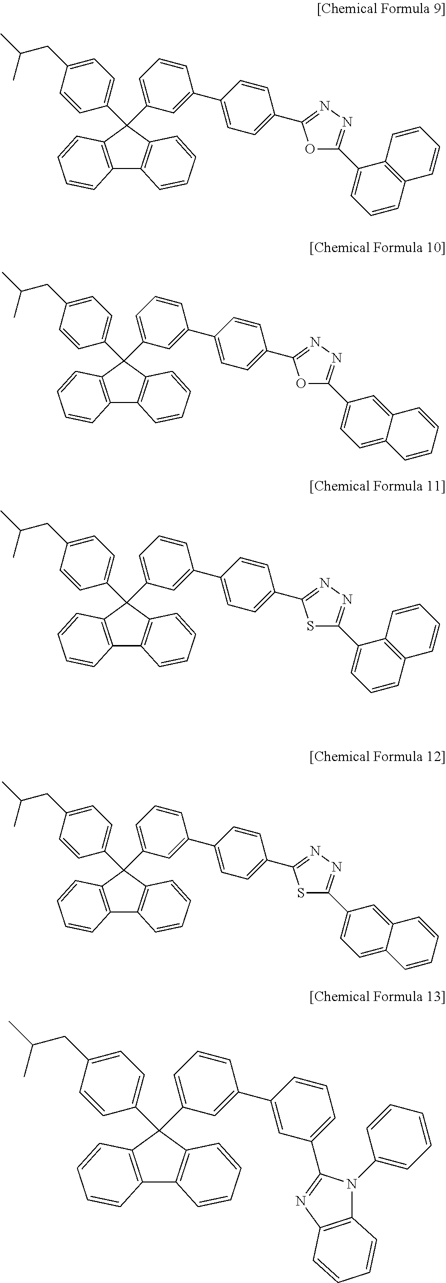

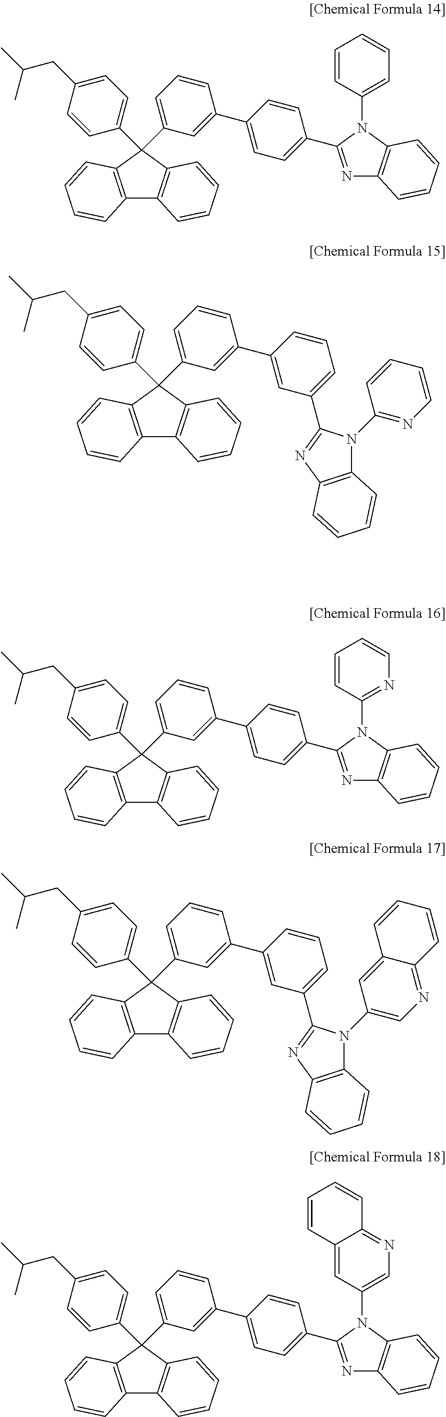

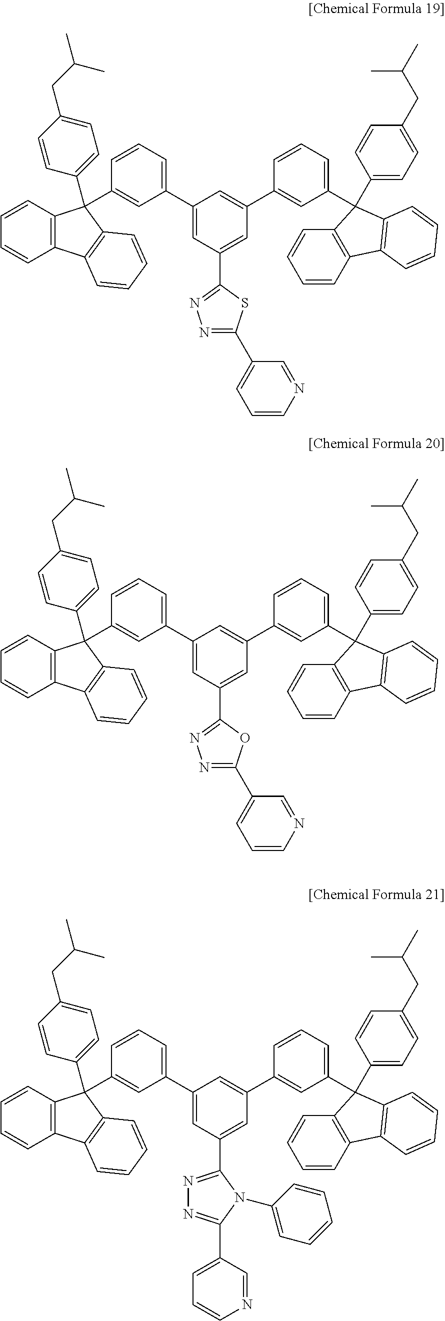

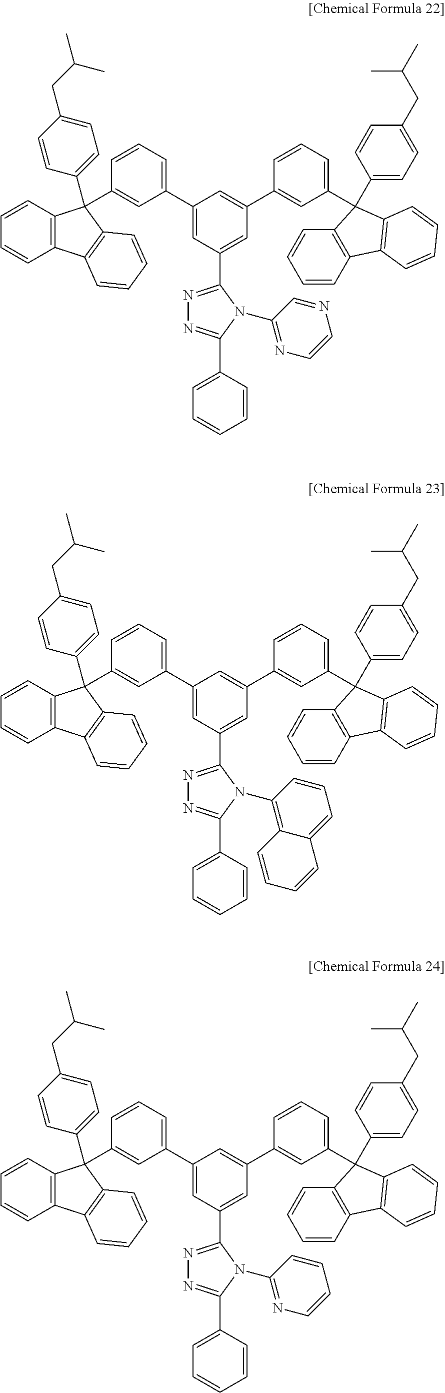

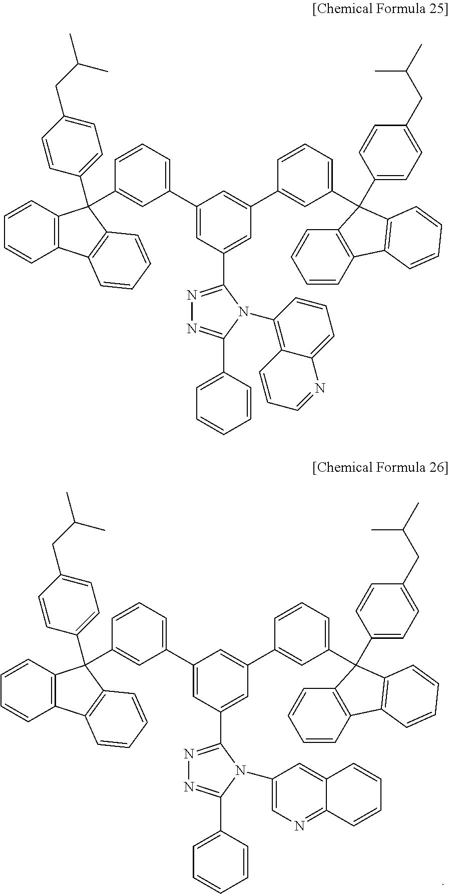

The compound may have a glass transition temperature (Tg) of about 110° C. or more and a thermal decomposition temperature (Td) of about 400° C. or more.

The embodiments may also be realized by providing an organic light emitting diode including an anode; a cathode; and at least one organic thin layer between the anode and cathode, wherein the at least one organic thin layer includes the compound for an organic optoelectronic device according to an embodiment.

The compound for an organic optoelectronic device may be a host material or a charge transport material.

The at least one organic thin layer may include the compound for an organic optoelectronic device singularly or as a host material along with a dopant.

The dopant may be a phosphorescent dopant selected from the group of a red, green, blue, and white light emitting dopant.

The at least one organic thin layer may be selected from the group of an emission layer, a hole transport layer (HTL), a hole injection layer (HIL), an electron blocking layer, an electron transport layer (ETL), an electron injection layer (EIL), a hole blocking layer, and a combination thereof.

The at least one organic thin layer may further include a compound represented by the following Chemical Formula 27.

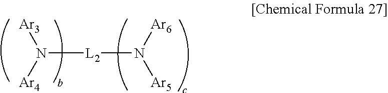

wherein, in Chemical Formula 27, Ar₃ to Ar₆ are each independently a substituted or unsubstituted C6 to C30 aryl group, L₂ is selected from the group of a substituted or unsubstituted C1 to C30 alkylene group, a substituted or unsubstituted C3 to C30 cycloalkylene group, a substituted or unsubstituted C6 to C30 arylene group, a substituted or unsubstituted C1 to C30 heteroarylene group, a substituted or unsubstituted C6 to C30 arylamine group, and a combination thereof, and b and c are each independently integers of 0 to 3, provided that b and c are not simultaneously 0.

The compound represented by Chemical Formula 27 may include a compound represented by one of the following Chemical Formulae 28 to 31.

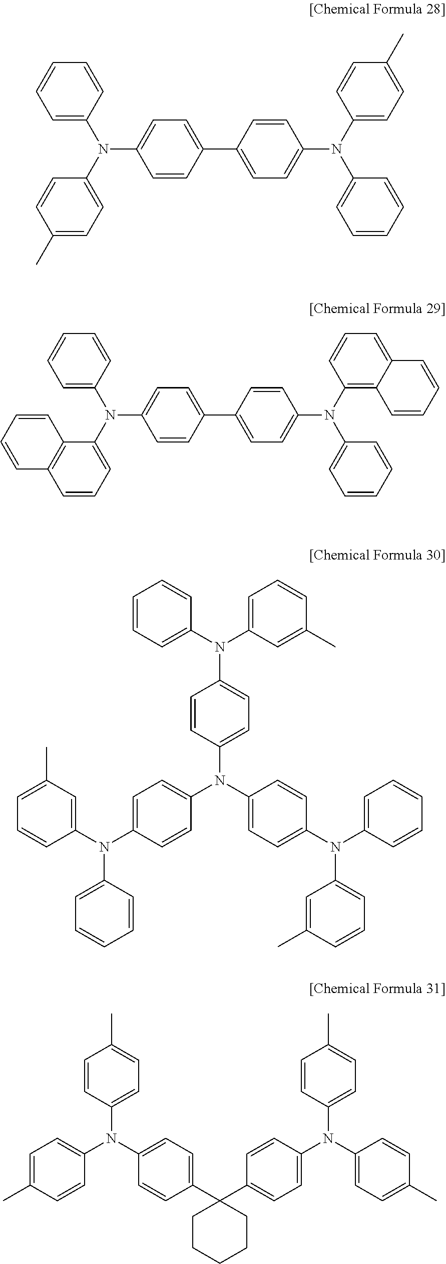

The compound for an organic optoelectronic device and the compound represented by Chemical Formula 27 may be included in the at least one organic thin layer in a weight ratio of about 99:1 to about 1:99.

The embodiments may also be realized by providing a display device comprising the organic light emitting diode according to an embodiment.

### BRIEF DESCRIPTION OF THE DRAWINGS

The embodiments will become more apparent to those of ordinary skill in the art by describing in detail exemplary embodiments with reference to the attached drawings, in which:

FIGS. 1 to 5 illustrate cross-sectional views of organic light emitting diode including compounds for an organic optoelectronic device according to various embodiments.

### DETAILED DESCRIPTION

Korean Patent Application No. 10-2008-0135689, filed on Dec. 29, 2008, in the Korean Intellectual Property Office, and entitled: “Novel Compounds for an Organic Photoelectric Device, and Organic Photoelectric Device Comprising Same,” is incorporated by reference herein in its entirety.

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey the scope of the invention to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. In addition, it will also be understood that when a layer is referred to as being “between” two layers, it can be the only layer between the two layers, or one or more intervening layers may also be present. Like reference numerals refer to like elements throughout.

As used herein, when specific definition is not otherwise provided, the term “substituted” may refer to one substituted with at least a substituent selected from the group of a C1 to C10 alkyl group, a C1 to C10 cycloalkyl group, a C6 to C20 aryl group, a C1 to C10 alkoxy group, a C6 to C20 arylamine group, a C2 to C20 heteroaryl group, and a cyano group.

As used herein, when specific definition is not otherwise provided, the prefix “hetero” may refer to one including 1 to 3 heteroatoms of N, O, S, or P, and remaining carbons in one ring.

In this specification, as used herein, when a definition is not otherwise provided, the term “heterocyclic group” may refer to one selected from the group of a C2 to C30 heteroaryl group, a C1 to C30 heterocycloalkyl group, a C1 to C30 heterocycloalkenyl group, and a C1 to C30 heterocycloalkynyl group, which includes a heteroatom. The heterocyclic group may include 1 to 15 heteroatoms.

An embodiment provides a compound for an organic optoelectronic device, the compound being represented by the following Chemical Formula 1.

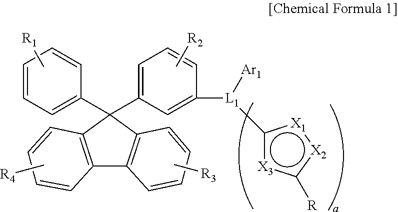

In Chemical Formula 1,

Ar₁ may be hydrogen or a substituted or unsubstituted C18 to C40 fluorenyl group,

L₁ may be selected from the group of a substituted or unsubstituted C1 to C30 alkylene group, a substituted or unsubstituted C6 to C30 arylene group, a substituted or unsubstituted C1 to C30 heteroarylene group, and N,

X₁ to X₃ may each independently be selected from the group of N, NR′₁, O, S, and CR′₂, provided that at least two of X₁ to X₃ are selected from the group of N, O, and S,

R, R′₁, and to R′₂ may each independently be a substituted or unsubstituted C1 to C30 alkyl group, a substituted or unsubstituted C1 to C30 alkylene group, a substituted or unsubstituted C2 to C30 alkenyl group, substituted or unsubstituted C2 to C30 alkenylene group, substituted or unsubstituted C6 to C30 aryl group, and substituted or unsubstituted C1 to C30 heteroaryl group. Optionally, adjacent two of R, R′₁, and to R₂ may form a fused ring.

a may be an integer of 1 to 4.

R₁ to R₄ may each independently be hydrogen or a substituted or unsubstituted C1 to C10 alkyl group.

In Chemical Formula 1, including a fluorenyl group substituent may increase solubility and thermal stability. In addition, a substituent including X₁ to X₃ in Chemical Formula 1 may be an electron transport substituent. Accordingly, the compound for an organic optoelectronic device may have good electron transporting characteristics and thus, may be used with a hole transport material to improve efficiency of an organic photoelectric device.

In an implementation, the compound for an organic optoelectronic device may include another fluorenyl group, thereby increasing solubility of the compound in an organic solvent. Thus, even though the compound may have a relatively low molecular weight, it may easily form an organic thin layer when using a wet process. Accordingly, Ar₁ in Chemical Formula 1 may include a substituted or unsubstituted fluorenyl group to regulate solubility of the compound in an organic solvent.

For example, the compound for an organic optoelectronic device (represented by Chemical Formula 1) may be represented by the following Chemical Formula 2.

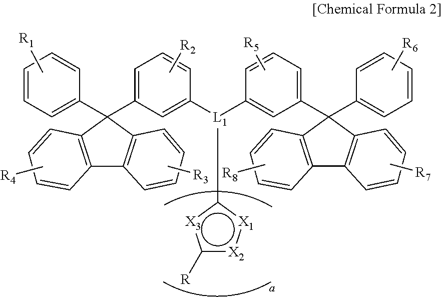

In Chemical Formula 2,

L₁ may be selected from the group of a substituted or unsubstituted C1 to C30 alkylene group, a substituted or unsubstituted C6 to C30 arylene group, a substituted or unsubstituted C1 to C30 heteroarylene group, and N,

X₁ to X₃ may each independently be selected from the group of N, NR′₁, O, S, and CR′₂, provided that at least two of X₁ to X₃ are selected from the group of N, O, and S.

R, R′₁, and to R′₂ may each independently be selected from the group of a substituted or unsubstituted C1 to C30 alkyl group, a substituted or unsubstituted C1 to C30 alkylene group, a substituted or unsubstituted C2 to C30 alkenyl group, a substituted or unsubstituted C2 to C30 alkenylene group, a substituted or unsubstituted C6 to C30 aryl group, and a substituted or unsubstituted C1 to C30 heteroaryl group. Optionally, adjacent two of R, R′₁, and to R′₂ may form a fused ring.

a may be an integer of 1 to 4.

R₁ to R₈ may each independently be hydrogen or a substituted or unsubstituted C1 to C10 alkyl group.

In Chemical Formulae 1 and 2, when a is an integer of 2 to 4, the substituent of R, R′₁, and to R′₂ may be independent (or different from each other).

The compound for an organic optoelectronic device according to an embodiment, e.g., represented by Chemical Formula 1, may include a compound represented by one of the following Chemical Formulae 3 to 26. However, the compound is not limited thereto.

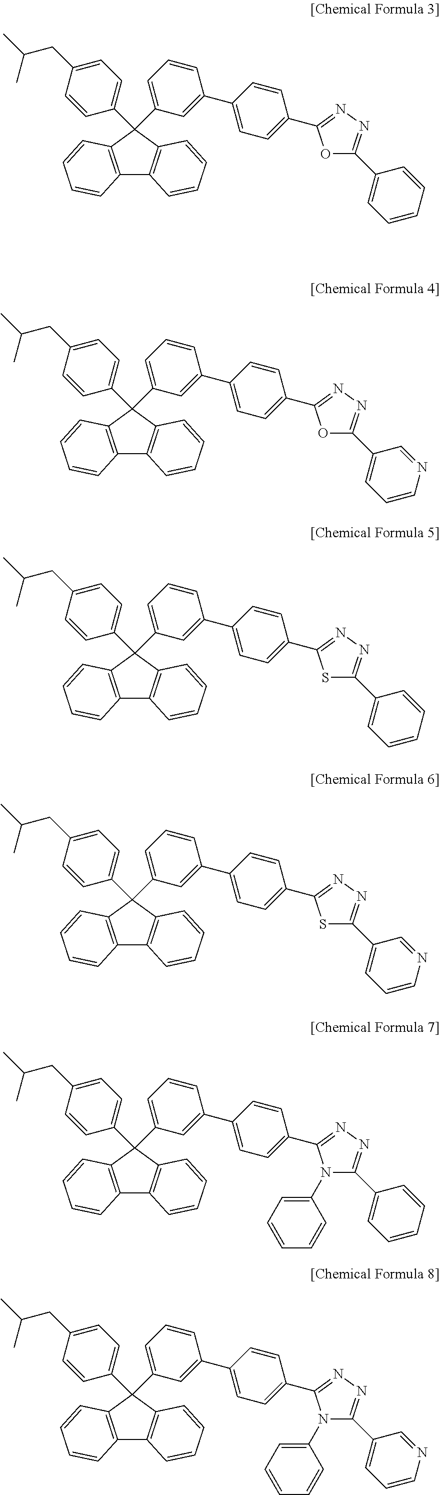

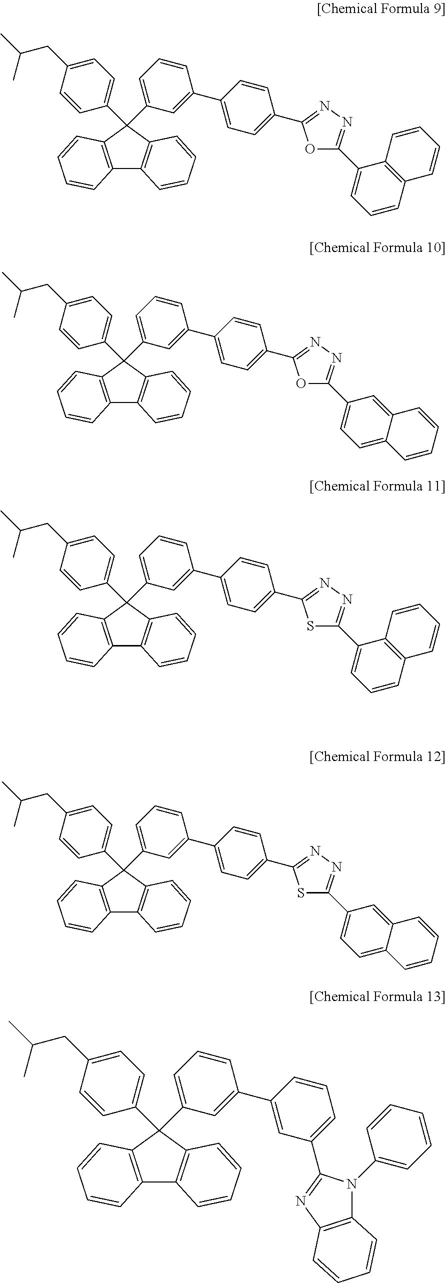

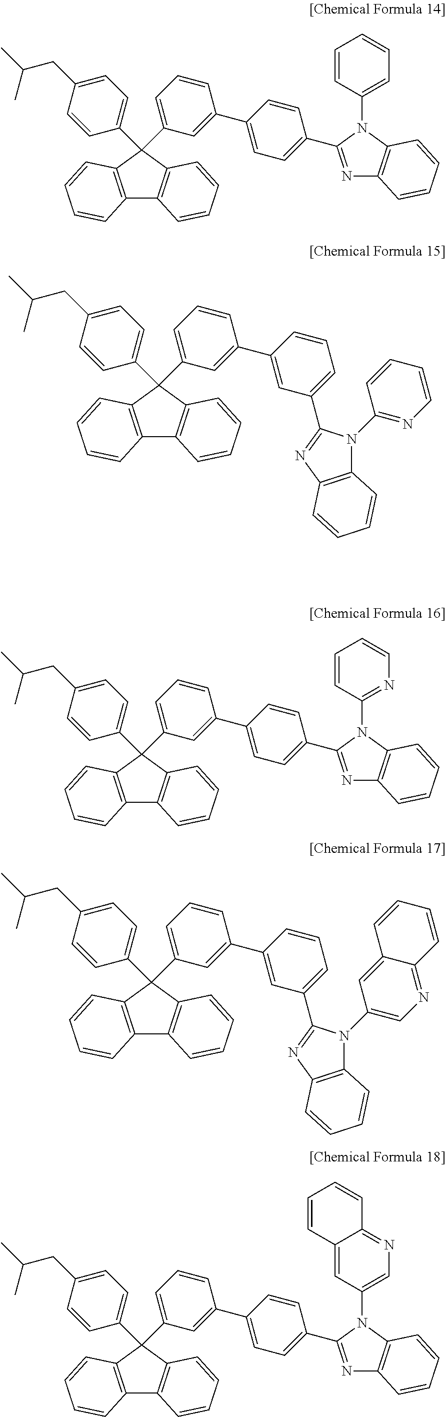

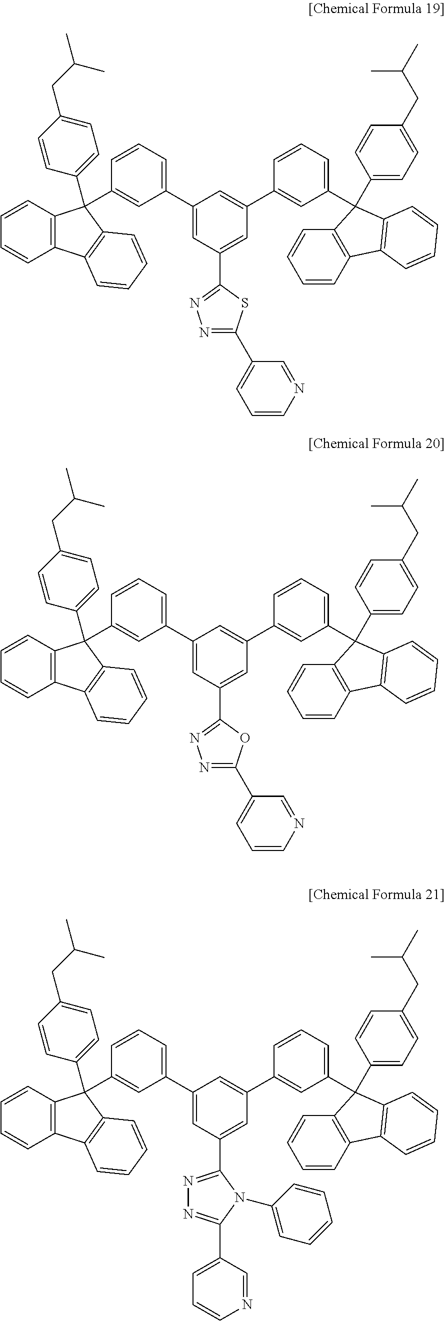

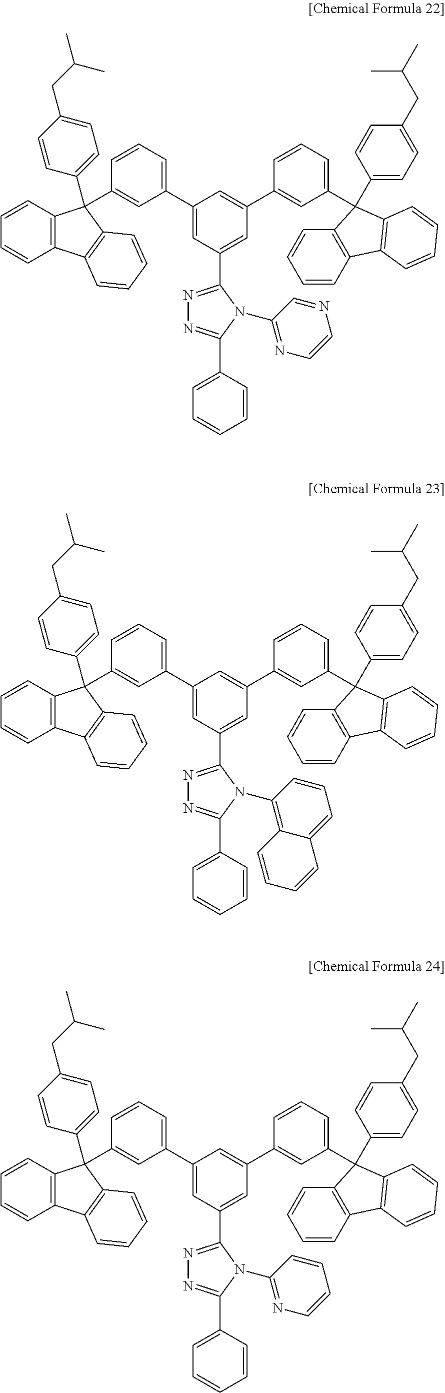

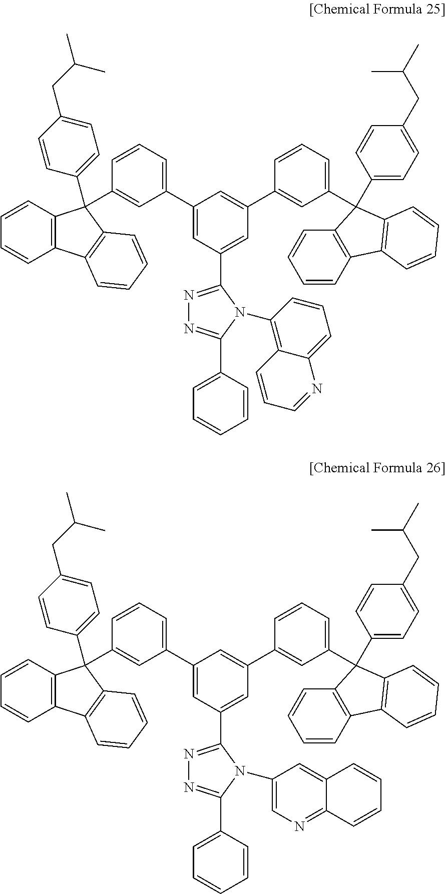

The compound for an organic optoelectronic device according to an embodiment may be used singularly or as a host material along with a dopant. The dopant may be a compound having a high emission property by itself. However, the dopant may be added to a host in a minor amount, so it may also be called a guest. The dopant may include a typical dopant without limitation. In an implementation, the dopant may include a fluorescent or phosphorescent dopant that has high light emitting quantum efficiency, is not agglomerated, and may be uniformly distributed in a host material.

For example, the dopant may be a phosphorescent dopant material emitting one selected from the group of red, green, blue, and white light, e.g., a metal complex capable of light-emitting by multiplet excitation such as triplet excitation or more. The phosphorescent dopant may be an organic metal compound including an element selected from the group of Ir, Pt, Os, Ti, Zr, Hf, Eu, Tb, Tm, Fe, Co, Ni, Ru, Rh, Pd, and a combination thereof. For example, a red phosphorescent dopant may include a platinum-octaethylporphine complex (PtOEP), Ir(Piq)₂(acac), Ir(Piq)₃, RD 61 from UDC, and the like, a green phosphorescent dopant may include Ir(PPy)₂(acac), Ir(PPy)₃, GD48 from UDC, and the like, and a blue phosphorescent dopant may include (4,6-F₂PPy)₂Irpic, and the like. The “Piq” denotes 1-phenylisoquinoline, “acac” denotes pentane-2,4-dione, “F2PPy” denotes 2-(difluorophenyl)pyridinato, “pic” denotes picolinate, and “PPy” denotes 2-phenylpyridine.

The compound for an organic optoelectronic device according to an embodiment may have a glass transition temperature (Tg) of about 110° C. or more and a thermal decomposition temperature (Td) of about 400° C. or more. For example, the glass transition temperature (Tg) may be about 110 to about 200° C.; and the thermal decomposition temperature (Td) may be about 400 to about 600° C. The compound may be used as a host material or charge transport material having thermal stability and electrochemical stability. In an implementation, the thermal decomposition temperature (Td) may be about 430° C. or more.

The compound for an organic optoelectronic device according to an embodiment may be applied to an organic thin layer of an organic photoelectric device. Accordingly, an organic photoelectric device that generates phosphorescence at a wide range of wavelength region from red to blue, has improved efficiency, and a low driving voltage may be provided. Also, life-span of the organic photoelectric device may be improved.

Thus, another embodiment provides an organic optoelectronic device including the compound for an organic optoelectronic device. Such an organic optoelectronic device may include an organic photoelectric device, an organic light emitting diode (OLED), an organic solar cell, an organic photo conductor drum, an organic transistor, an organic memory device, or the like. For example, the compound for an organic optoelectronic device according to an embodiment may be applied to an electrode or an electrode buffer layer of an organic solar cell to improve quantum efficiency, or may be applied to an electrode material of a gate, source-drain electrodes, and the like, of an organic transistor.

Hereinafter, the organic light emitting diode is described in more detail.

The organic light emitting diode according to an embodiment may include an anode, a cathode, and at least one organic thin layer between the anode and cathode. The organic thin layer may include the compound for an organic optoelectronic device according to an embodiment.

The compound for an organic optoelectronic device may be included in an organic thin layer that includes, e.g., an emission layer, a hole transport layer (HTL), a hole injection layer (HIL), a hole blocking layer, an electron transport layer (ETL), an electron injection layer (EIL), an electron blocking layer, or a combination thereof. At least one of these organic thin layers may include the compound for an organic optoelectronic device. For example, at least one of the emission layer, hole transport layer (HTL), hole injection layer (HIL), electron transport layer (ETL), electron injection layer (EIL), or a combination thereof may include the compound for an organic optoelectronic device according to an embodiment.

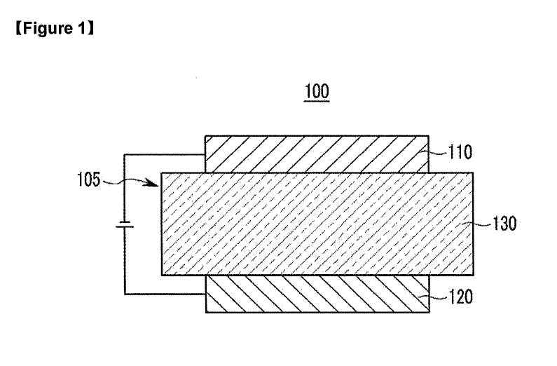

FIGS. 1 to 5 illustrate cross-sectional views of organic light emitting diodes including the organic compounds according to various embodiments.

Referring to FIGS. 1 to 5, the organic light emitting diodes 100, 200, 300, 400, and 500 according to an embodiment may include at least one organic thin layer 105 between an anode 120 and a cathode 110.

A substrate of the organic light emitting diode is not particularly limited, and may include a glass substrate or a transparent plastic substrate having excellent transparency, surface smoothness, handling ease, and water repellency.

The anode 120 may include an anode material laving a large work function to facilitate hole injection into an organic thin layer. The anode material may include a metal such as nickel, platinum, vanadium, chromium, copper, zinc, and gold, or alloys thereof; a metal oxide such as zinc oxide, indium oxide, indium tin oxide (ITO), and indium zinc oxide (IZO); a combined metal and oxide such as ZnO:Al or SnO₂:Sb; or a conductive polymer such as poly(3-methylthiophene), poly[3,4-(ethylene-1,2-dioxy)thiophene] (PEDT), polypyrrole, and polyaniline. But the anode material is not limited there to. For example, the anode material may preferably be a transparent electrode including ITO.

The cathode 110 may include a cathode material having a small work function to facilitate electron injection into an organic thin layer. The cathode material may include a metal such as magnesium, calcium, sodium, potassium, titanium, indium, yttrium, lithium, gadolinium, aluminum, silver, tin, and lead, or alloys thereof; or a multi-layered material such as LiF/Al, Liq/Al, L₁O₂/Al, LiF/Ca, LiF/Al, and BaF₂/Ca. But the cathode material is not limited to the above compound. For example, the cathode material may preferably be a metal electrode such as aluminum.

Referring to FIG. 1, the organic light emitting diode 100 may include an organic thin layer 105 including only an emission layer 130.

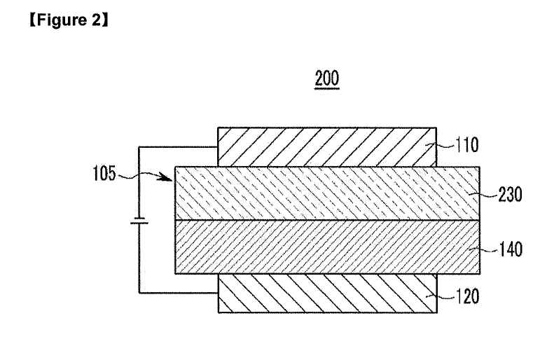

Referring to FIG. 2, a double-layered organic light emitting diode 200 may include an organic thin layer 105 including an emission layer 230 including an electron transport layer (ETL) and a hole transport layer (HTL) 140. The emission layer 130 may also function as an electron transport layer (ETL); and the hole transport layer (HTL) 140 layer may have an excellent binding property with a transparent electrode, e.g., an ITO electrode, and/or an excellent hole transporting property.

The hole transport layer (HTL) 140 may be a typical HTL without limitation, e.g., poly(3,4-ethylenedioxy-thiophene) (PEDOT) doped with poly(styrenesulfonate) (PSS) (PEDOT:PSS), N,N′-bis(3-methylphenyl)-N,N-diphenyl-[1,1′-biphenyl]-4,4′-diamine (TPD), or the like, along with the compound for an organic optoelectronic device according to an embodiment.

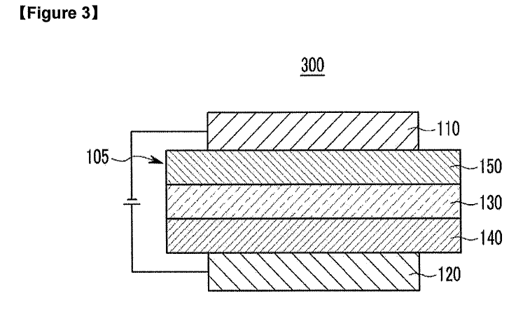

Referring to FIG. 3, a three-layered organic light emitting diode 300 may include an organic thin layer 105 including an electron transport layer (ETL) 150, an emission layer 130, and a hole transport layer (HTL) 140. The emission layer 130 may be independently installed, and layers having an excellent electron transporting property or an excellent hole transporting property may be separately stacked.

The electron transport layer (ETL) 150 may include a typical ETL without limitation, e.g., aluminum tris(8-hydroxyquinoline) (Alq₃), a 1,3,4-oxadiazole derivative such as 2-(4-biphenyl-5-phenyl-1,3,4-oxadiazole (PBD); a quinoxalin derivative such as 1,3,4-tris[(3-phenyl-6-trifluoromethyl)quinoxalin-2-yl]benzene (TPQ); and a triazole derivative, along with the compound for an organic optoelectronic device according to an embodiment.

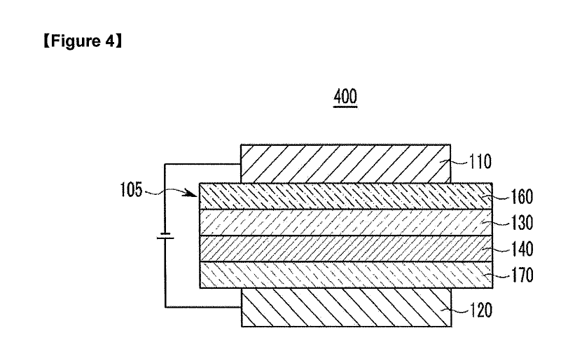

As shown in FIG. 4, a four-layered organic light emitting diode 400 may include an organic thin layer 105 including an electron injection layer (EIL) 160, an emission layer 130, a hole transport layer (HTL) 140, and a hole injection layer (HIL) 170 (for binding with the anode 120 of, e.g., ITO).

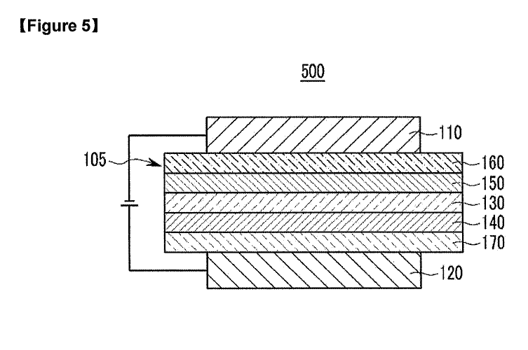

As shown in FIG. 5, a five layered organic light emitting diode 500 may include an organic thin layer 105 including an electron transport layer (ETL) 150, an emission layer 130, a hole transport layer (HTL) 140, and a hole injection layer (HIL) 170, and may further include an electron injection layer (EIL) 160 to achieve a low voltage.

The emission layers 130 and 230 may have a thickness of about 5 to about 1,000 nm; and the hole transport layer (HTL) 140 and electron transport layer (ETL) 150 may have a thickness of about 10 to about 10,000 Å, respectively. However, the thicknesses are not limited to the above range.

In FIGS. 1 to 5, at least one of the organic thin layer 105 selected from the group of the electron transport layer (ETL) 150, electron injection layer (EIL) 160, emission layer 130 and 230, hole transport layer (HTL) 140, hole injection layer (HIL) 170, and a combination thereof may include the compound for an organic optoelectronic device according to an embodiment. An organic thin layer, e.g., a hole blocking layer and an electron blocking layer, may include the compound to limit transfer speed of holes or electrons in an emission layer to thereby increase combination population of electrons and holes.

For example, the compound for an organic optoelectronic device may be preferably used in the emission layers 130 and 230.

The organic light emitting diode may be fabricated by forming an anode on a substrate, forming an organic thin layer, and forming a cathode thereon. The organic thin layer may be formed by a dry coating method, e.g., evaporation, sputtering, plasma plating, and ion plating; or a wet coating method, e.g., spin coating, dipping, flow coating, ink-jet printing, and the like.

For example, when an organic thin layer of an organic light emitting diode according to an embodiment is formed using a wet coating method, a compound represented by the following Chemical Formula 27 may be further included.

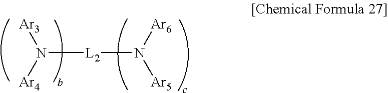

In Chemical Formula 27,

Ar₃ to Ar₆ may each independently be a substituted or unsubstituted C6 to C30 aryl group,

L₂ may be selected from the group of a substituted or unsubstituted C1 to C30 alkylene group, a substituted or unsubstituted C3 to C30 cycloalkylene group, a substituted or unsubstituted C6 to C30 arylene group, a substituted or unsubstituted C1 to C30 heteroarylene group, a substituted or unsubstituted C6 to C30 arylamine group, and a combination thereof, and

b and c may each independently be integers of 0 to 3, provided that b and c are not simultaneously 0.

In an implementation, the compound represented by Chemical Formula 27 may be a compound represented by one of the following Chemical Formulae 28 to 31.

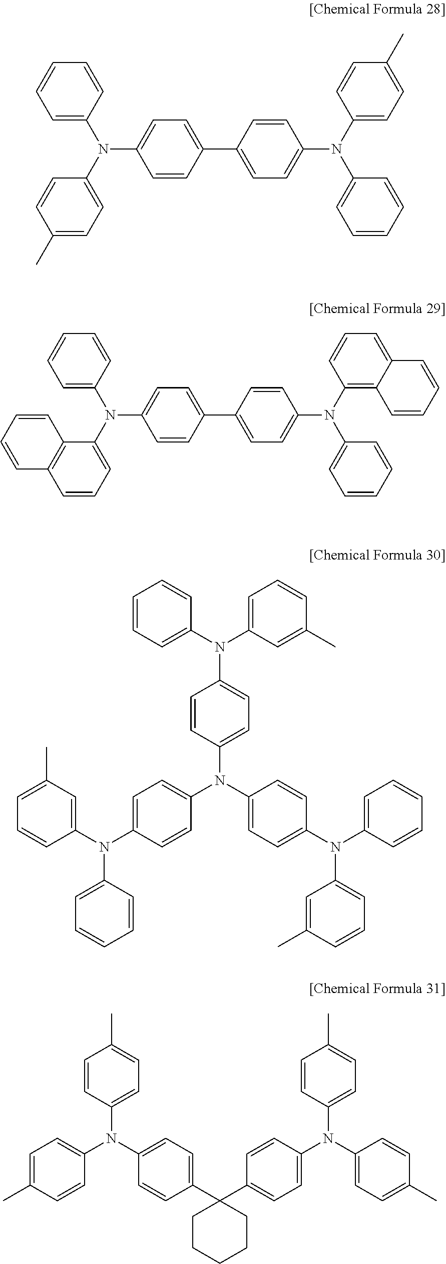

In an implementation, the compound for an organic optoelectronic device according to an embodiment and the compound of Chemical Formula 27 may be used or included at a weight ratio of about 1:99 to about 99:1.

The compound according to an embodiment may be blended with polymers having conjugated double bonds such as fluorene-based, polyphenylenevinylene-based, polyparaphenylene-based polymers, or mixed with binder resins. The binder resin may be a typical binder resin without limitation, and may include a resin of polyvinylcarbazole, polycarbonate, polyester, polyacrylate, polystyrene, a (meth)acryl-based polymer, polybutyral, polyvinylacetal, a diallylphthalate polymer, a phenol resin, an epoxy resin, a silicone resin, a polysulfone resin, a urea resin, and the like, or a mixture of two or more.

Another embodiment provides a display device including the organic light emitting diode according to the above-described embodiment.

The following Examples and Comparative Examples are provided in order to set forth particular details of one or more embodiments. However, it will be understood that the embodiments are not limited to the particular details described. Further, the Comparative Examples are set forth to highlight certain characteristics of certain embodiments, and are not to be construed as either limiting the scope of the invention as exemplified in the Examples or as necessarily being outside the scope of the invention in every respect.

Synthesis of Compound for Organic Optoelectronic Device

### Example 1

### Synthesis of Chemical Formula 3 Compound

A compound represented by the above Chemical Formula 3 (as a compound for an organic optoelectronic device according) was synthesized through the following one step as shown in Reaction Scheme 1.

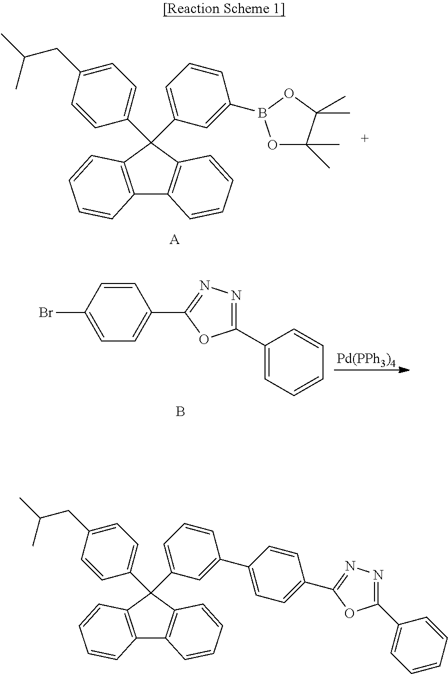

5 g (10 mmol) of a compound A and 3.3 g (11 mmol) of a compound B were dissolved in 100 mL of tetrahydrofuran in a 250 mL round-bottomed flask with a reflux condenser and an agitator under a nitrogen atmosphere; and 80 mL of a 2 M potassium carbonate aqueous solution was mixed therewith. Then, 0.23 g (0.2 mmol) of tetrakis(triphenylphosphine) palladium (Pd(PPh₃)₄) was added to the mixed solution. The resulting mixture was refluxed.

When the reaction was complete, the reactant was extracted several times using methylene chloride. Then, anhydrous magnesium sulfate was used to remove moisture from the extract; and the solvent was removed therefrom. Next, the reactant was purified through a silica gel chromatography, obtaining 4.3 g (yield: 73%) of a compound represented by Chemical Formula 3. Atomic analysis of the obtained compound represented by Chemical Formula 3 was performed. The result is provided as follows.

calcd. C₄₃H₃₄N₂O: C, 86.84; H, 5.76; N, 4.71. found: C, 86.02; H, 5.34; N, 4.65.

### Example 2

### Synthesis of a Compound of Chemical Formula 13

A compound represented by the above Chemical Formula 13 (as a compound for an organic optoelectronic device) was synthesized through the following one step as shown in Reaction Scheme 2.

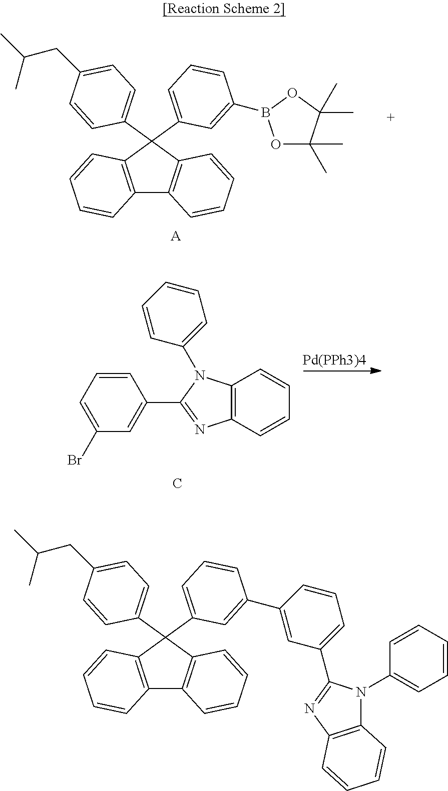

5 g (10 mmol) of a compound A and 3.8 g (11 mmol) of a compound C were dissolved in 100 mL of tetrahydrofuran in a 250 mL round-bottomed flask with a reflux condenser and an agitator under a nitrogen atmosphere, and 80 mL of a 2 M potassium carbonate aqueous solution was added thereto. Then, 0.23 g (0.2 mmol) of tetrakis(triphenylphosphine) palladium (Pd(PPh₃)₄) was added to the mixed solution. The resulting mixture was refluxed.

When the reaction was complete, the reactant was extracted several times using methylene chloride. Then, anhydrous magnesium sulfate was used to remove moisture from the extract, and the solvent was removed therefrom. Then, the reactant was purified using a silica gel chromatography, obtaining a 4.1 g (yield: 65%) of a compound represented by Chemical Formula 13. Atomic analysis of the obtained compound represented by Chemical Formula 13 was performed. The result is provided as follows.

calcd. C₄₈H₃₈N₂: C, 89.68; H, 5.96; N, 4.36. found: C, 89.13; H, 5.58; N, 4.11.

### Example 3

### Synthesis of a Compound of Chemical Formula 20

A compound represented by the above Chemical Formula 20 (as a compound for an organic optoelectronic device) was synthesized through the following one step as shown in Reaction Scheme 3.

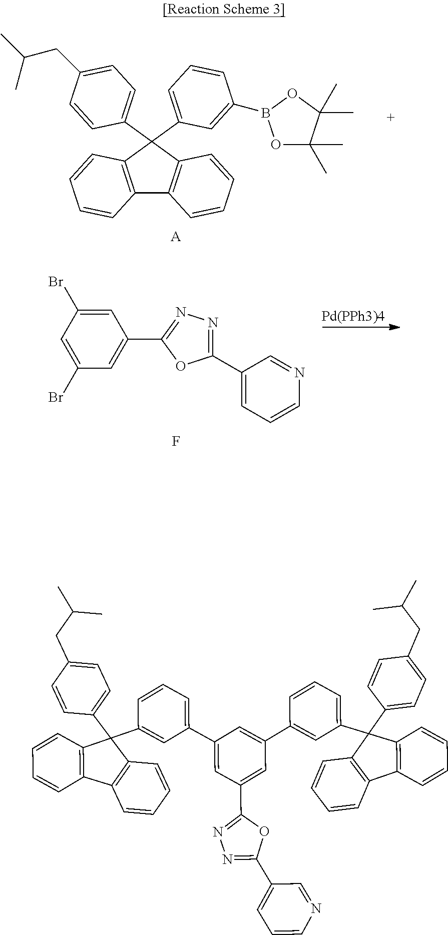

1 g (2.6 mmol) of a compound F and 3.3 g (6.6 mmol) of a compound A were dissolved in 100 mL of tetrahydrofuran in a 250 mL round-bottomed flask with a thermostat, a reflux condenser, and an agitator, and 80 mL of a 2 M potassium carbonate aqueous solution was added thereto. Then, 0.06 g (0.05 mmol) of tetrakis(triphenylphosphine) palladium (Pd(PPh₃)₄) was added to the mixed solution.

When the reaction was complete, the reactant was extracted several times using methylene chloride. Then, anhydrous magnesium sulfate was used to remove moisture from the extract, and the solvent was removed therefrom. Next, the reactant was purified using a silica gel chromatography, obtaining 1.5 g (yield: 60%) of a compound represented by Chemical Formula 20. Atomic analysis of the obtained compound represented by Chemical Formula 20 was performed. The result is provided as follows.

calcd. C71H₅₇N₃O: C, 88.07; H, 5.93; N, 4.34. found: C, 87.85; H, 5.31; N, 4.04.

Fabrication of an Organic Light Emitting Diode

### Example 4

An ITO substrate was used as an anode; and poly(3,4-ethylene dioxy-thiophene) (PEDOT) was spin-coated on the substrate.

On the PEDOT, an emission layer was spin-coated. The emission layer was formed by using (as a host) a mixture of the compound represented by Chemical Formula 3 prepared in Example 1 and the compound represented by the following Chemical Formula 30 at a weight ratio of 1:1 and Ir(piq)₂(acac) (as a dopant). Herein, the Ir(piq)₂(acac) dopant was included in an amount of 7 wt %, based on 100 wt % of the total weight of an emission layer.

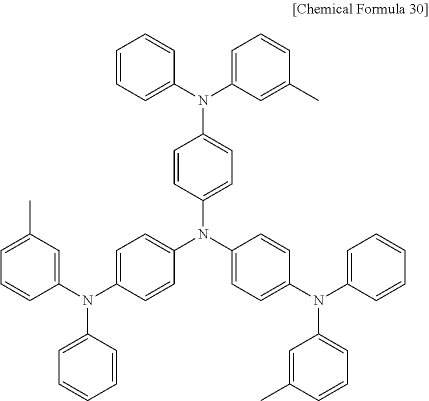

Next, a hole blocking layer was formed by vacuum-deposition of bis(8-hydroxy-2-methylquinolinolato)-aluminumbiphenoxide (BAlq) to be 50 Å thick on the emission layer. In addition, Alq₃ was vacuum-deposited to be 200 Å thick on the hole blocking layer to form an electron transport layer (ETL).

On the electron transport layer (ETL), LiF and Al were sequentially vacuum-deposited to be respectively 10 Å and 1,000 Å to form a cathode, completing an organic light emitting diode.

The organic light emitting diode had a structure as follows.

Al 1,000 Å/LiF 10 Å/Alq₃ 200 Å/BAlq 50 Å/EML (the compound of Chemical Formula 3: the compound of Chemical Formula 30 a weight ratio of 50:50)+Ir(piq)₂(acac) (7 wt %)) 500 Å/PEDOT 400 Å/ITO 1,500 Å

### Example 5

An organic light emitting diode was fabricated according to the same method as in Example 4 except for using the compound of Chemical Formula 13 (instead of the compound of Chemical Formula 3) in the host for the emission layer.

The organic light emitting diode had a structure as follows.

Al 1,000 Å/LiF 10 Å/Alq₃ 200 Å/BAlq 50 Å/EML (the compound of Chemical Formula 13: the compound of Chemical Formula 30 (a weight ratio of 50:50)+Ir(piq)₂(acac) (7 wt %)) 500 Å/PEDOT 400 Å/ITO 1,500 Å

### Example 6

An organic light emitting diode was fabricated according to the same method as in Example 4 except for using the compound of Chemical Formula 20 (instead of the compound of Chemical Formula 3) in the host for the emission layer.

The organic light emitting diode had a structure as follows.

Al 1,000 Å/LiF 10 Å/Alq₃ 200 Å/BAlq 50 Å/EML (the compound of Chemical Formula 20: the compound of Chemical Formula 30 (a weight ratio of 50:50)+Ir(piq)₂(acac) (7 wt %)) 500 Å/PEDOT 400 Å/ITO 1,500 Å

### Comparative Example 1

An organic light emitting diode was fabricated according to the same method as in Example 4 except for using 1,3,5-tris(N-phenylbenzimidazol-2-yl)benzene (TPBI) (instead of the compound of Chemical Formula 3) in the host for the emission layer.

The organic light emitting diode had a structure as follows.

Al 1,000 Å/LiF 10 Å/Alq₃ 200 Å/BAlq 50 Å/EML (TPBI: a compound of Chemical Formula 30 (in a weight ratio of 50:50)+Ir(piq)₂(acac) (7 wt %)) 500 Å/PEDOT 400 Å/ITO 1,500 Å

### Experimental Example 1

### Evaluation of Organic Light Emitting Diodes

(1) Measurement of Current Density Change Depending on Voltage Change

The organic light emitting diodes were measured regarding a current flowing in a unit device while voltage was increased from 0 V to 10 V by using a current-voltage meter (Keithley 2400). The current value was divided by an area to calculate current density.

(2) Measurement of Luminance Change Depending on Voltage Change

The organic light emitting diodes were measured regarding luminance while voltage is increased from 0 V to 10 V by using a luminance meter (Minolta Cs-1000A).

(3) Measurement of Luminous Efficiency

The luminance and current density and voltage measured in the above 1 and 2 were used to calculate current efficiency (cd/A) and electric power efficiency (lm/W) at the same luminance (1,000 cd/m²). The result is provided in the following Table 1.

(4) Color coordinates were measured using a luminance meter (Minolta Cs-100a). The result is provided in the following Table 1.

(5) Life-spans were measured by flowing a predetermined current in a device to emit luminance of 1,000 nit and then, measuring how long it took for luminance to decrease down to 50%. The result is provided in the following Table 2.

TABLE 1

| | | At 1,000 nit | | | |
|---|---|---|---|---|---|
| | | | Current | Electrical | |
| | | Driving | effici- | power | Color |
| | Host material of | voltage | ency | efficiency | coordinate |
| Device | emission layer | (V) | (cd/A) | (lm/W) | (x, y) |
| Comp. | Chemical Formula | 8.2 | 3.9 | 1.5 | 0.68, 0.32 |
| Ex. 1 | 30/TPBI | | | | |
| Ex. 4 | Chemical Formula | 6.9 | 5.3 | 2.4 | 0.68, 0.32 |
| | 30/Compound 3 | | | | |
| Ex. 6 | Chemical Formula | 6.9 | 5.9 | 2.7 | 0.68, 0.32 |
| | 30/Compound 20 | | | | |

TABLE 2

| Devices | Host material of emission layer | Life-span (hour) |
|---|---|---|
| Comp. Ex. 1 | Chemical Formula 30/TPBI | 66 |
| Ex. 4 | Chemical Formula 30/Compound 3 | 85 |
| Ex. 5 | Chemical Formula 30/Compound 13 | 72 |

Referring to Table 1, the organic light emitting diodes according to Examples 4 and 6 had lower driving voltage and much improved current efficiency and electric power efficiency and, resultantly, much improved device performance, compared with Comparative Example 1, based on the evaluation result.

In addition, referring to Table 2, the organic light emitting diodes according to Examples 4 and 5 were identified to have excellent life-span characteristic compared with TPBI (known as a good electron transport material).

By way of summation and review, a light emitting diode may have various efficiencies and performances depending on kinds of a host material used in an emission layer. In addition, a dopant (along with the host material) may be included in the emission layer to increase luminous efficiency and stability through color purity increase and energy transfer. For example, a low molecular weight host material, e.g., 4-N,N-dicarbazolebiphenyl (CBP), may be used as a host material, but may have very low thermal stability. In addition, the compound may have a high degree of symmetry and may be easily crystallized. Thus, when a device including the compound becomes hotter, a short-circuit or a pixel defect may occur. Furthermore, holes may generally be transported faster than electrons. Thus, an exciton may not be effectively formed in an emission layer, decreasing luminous efficiency of a device.

A low molecular weight host material may generally be applied using a vacuum-deposition. However, the vacuum deposition may cost more than a wet process. In addition, most low molecular weight host materials may have low solubility in an organic solvent and thus may not be applied using a wet process. Accordingly, it may be difficult to form an organic thin layer having excellent film characteristics.

Accordingly, the embodiments provide a phosphorescent host material and a charge transport material having excellent electrical and thermal stability and bipolar characteristics well-transporting both holes and electrons or a host material mixed with a material being able to well transport holes and electrons in order to realize an organic photoelectric device with excellent efficiency and life-span.

The embodiments provide a compound for an organic optoelectronic device having excellent electron transport capability.

The embodiments also provide an organic light emitting diode including the compound for an organic optoelectronic device, the organic light emitting diode having excellent life-span, efficiency, electrochemical stability, and thermal stability.

A compound for an organic optoelectronic device according to an embodiment may have high solubility in an organic solvent and may easily form an organic thin layer in a wet process, despite its low molecular weight. In addition, the compound for an organic optoelectronic device may emit phosphorescence in a wide wavelength region from red to blue, thereby achieving excellent efficiency.

Therefore, a compound for an organic optoelectronic device according to an embodiment may have excellent electrochemical and thermal stability. Thus, the compound may be included in an organic thin layer selected from the group of an emission layer, a hole transport layer (HTL), a hole injection layer (HIL), an electron blocking layer, an electron transport layer (ETL), an electron injection layer (EIL), a hole blocking layer, and a combination thereof. The compound may be used to prepare an organic optoelectronic device (such as an organic light emitting diode) having excellent life-span and high luminous efficiency at a low driving voltage.

Accordingly, the embodiments provide a compound for an organic optoelectronic device, the compound having excellent electron transport capability and thermal stability, being capable of easily forming an organic thin layer through a wet process, having excellent efficiency due to phosphorescent light emission, and being capable of providing an organic photoelectric device having excellent electrochemical stability, thermal stability, and life-span.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by the following Chemical Formula 1: 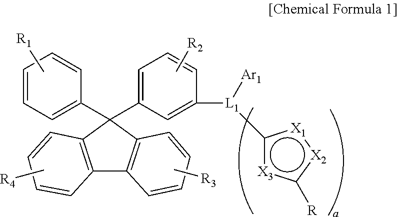
wherein, in Chemical Formula 1,
Ar₁ is hydrogen or a substituted or unsubstituted C18 to C40 fluorenyl group,
L₁ is selected from the group of a substituted or unsubstituted C1 to C30 alkylene group, a substituted or unsubstituted C6 to C30 arylene group, a substituted or unsubstituted C1 to C30 heteroarylene group, and N,
X₁ to X₃ are each independently selected from the group of N, NR′₁, O, S, and CR′₂, provided that at least two of X₁ to X₃ are selected from the group of N, O, and S,
R, R′₁, and to R′₂ are each independently selected from the group of hydrogen, a substituted or unsubstituted C1 to C30 alkyl group, a substituted or unsubstituted C1 to C30 alkylene group, a substituted or unsubstituted C2 to C30 alkenyl group, a substituted or unsubstituted C2 to C30 alkenylene group, a substituted or unsubstituted C6 to C30 aryl group, and a substituted or unsubstituted C1 to C30 heteroaryl group, and optionally adjacent two of R, R′₁, and to R′₂ form a fused ring,
a is an integer of 1 to 4, and
R₁ to R₄ are each independently hydrogen or a substituted or unsubstituted C1 to C10 alkyl group.

2. The compound for an organic optoelectronic device as claimed in claim 1, wherein the compound represented by Chemical Formula 1 includes a compound represented by the following Chemical Formula 2: 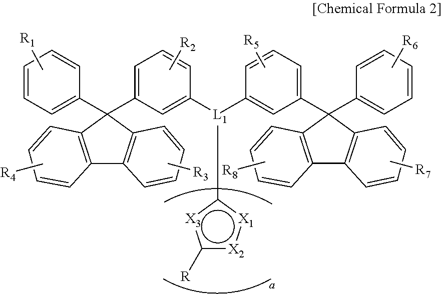
wherein in Chemical Formula 2,
L₁ is selected from the group of a substituted or unsubstituted C1 to C30 alkylene group, a substituted or unsubstituted C6 to C30 arylene group, a substituted or unsubstituted C1 to C30 heteroarylene group, and N,
X₁ to X₃ are each independently selected from the group of N, NR′₁, O, S, and CR′₂, provided that at least two of X₁ to X₃ are selected from the group of N, O, and S,
R, R′₁, and to R′₂, are each independently selected from the group of hydrogen, a substituted or unsubstituted C1 to C30 alkyl group, a substituted or unsubstituted C1 to C30 alkylene group, a substituted or unsubstituted C2 to C30 alkenyl group, a substituted or unsubstituted C2 to C30 alkenylene group, a substituted or unsubstituted C6 to C30 aryl group, and a substituted or unsubstituted C1 to C30 heteroaryl group, and optionally adjacent two of R, R′₁, and to R′₂ form a fused ring,
a is an integer of 1 to 4, and
R₁ to R₈ are each independently hydrogen or a substituted or unsubstituted C1 to C10 alkyl group.

3. The compound for an organic optoelectronic device as claimed in claim 1, wherein the compound represented by Chemical Formula 1 includes a compound represented by one of the following Chemical Formulae 3 to 26:

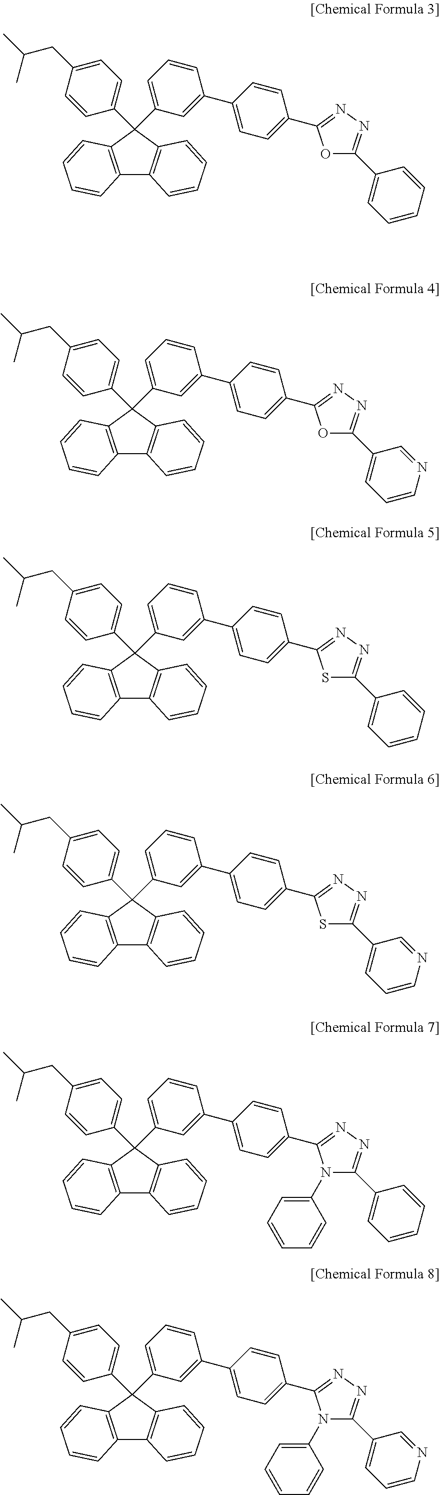

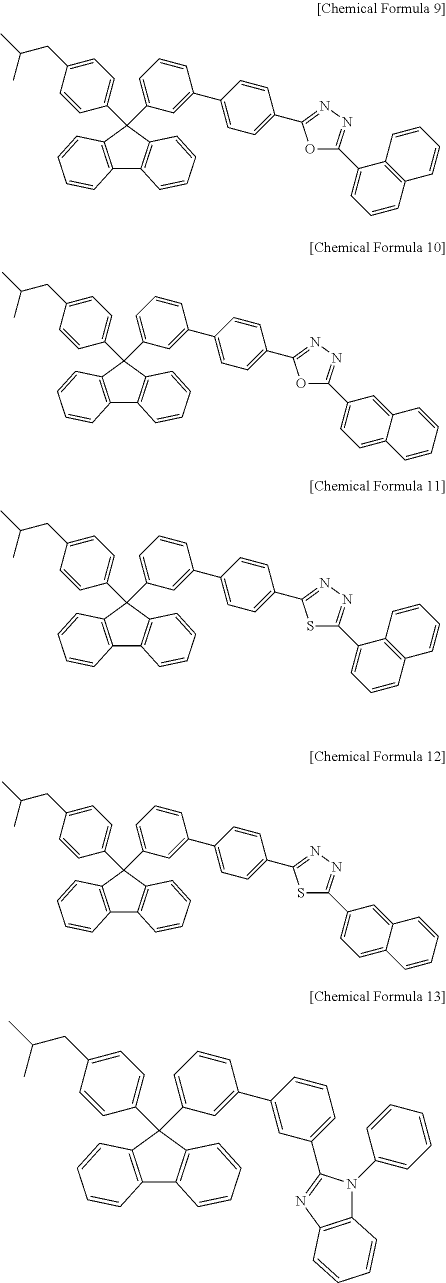

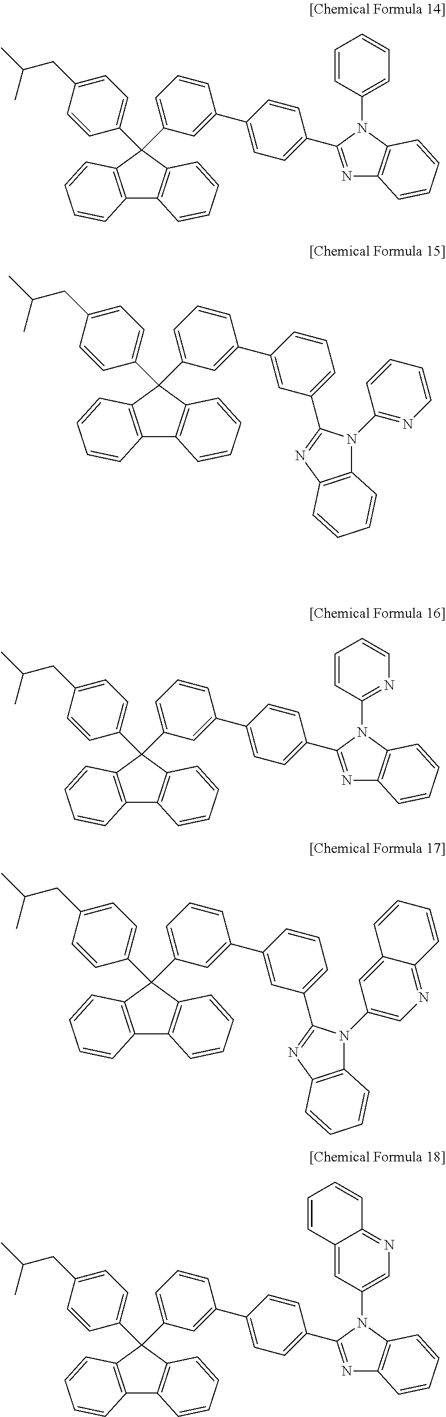

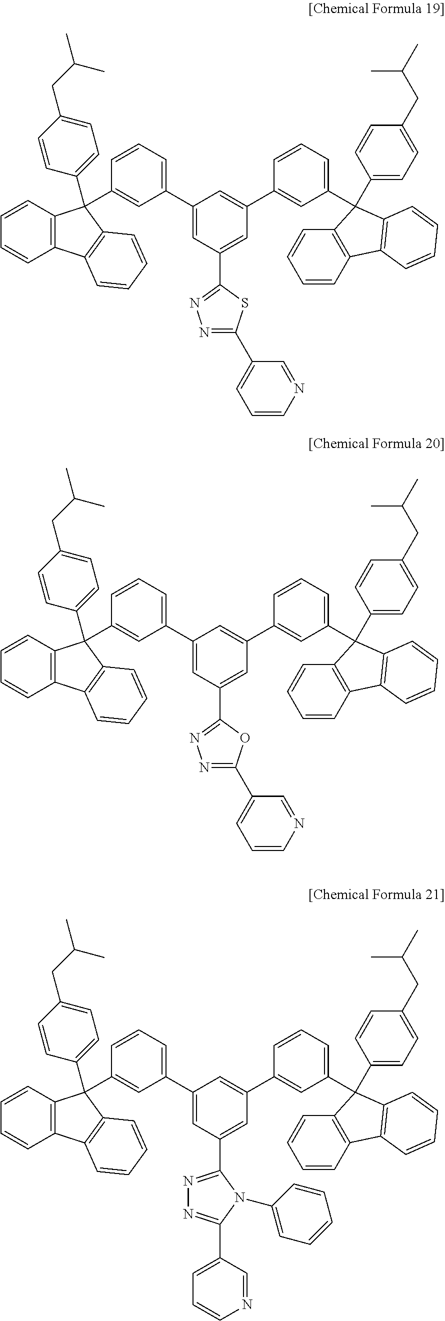

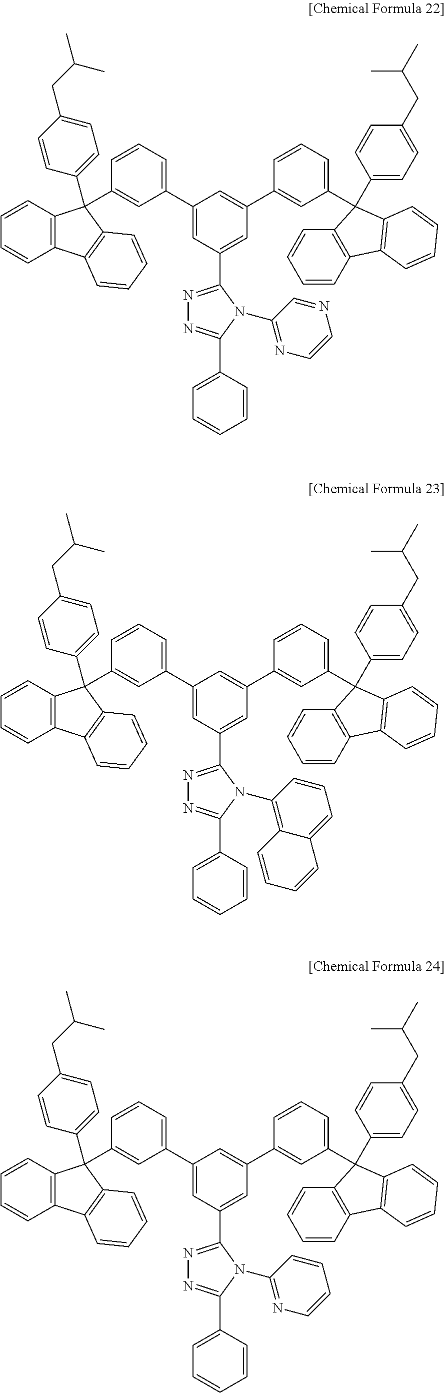

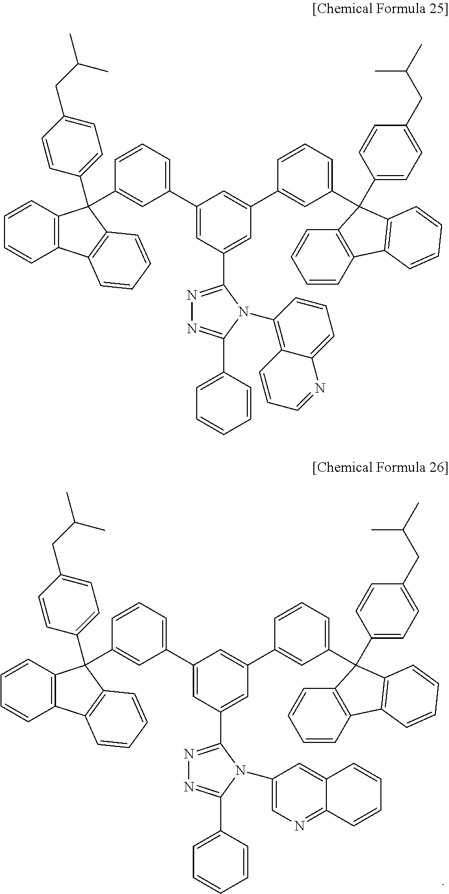

4. The compound for an organic optoelectronic device as claimed in claim 1, wherein the compound has a glass transition temperature (Tg) of about 110° C. or more and a thermal decomposition temperature (Td) of about 400° C. or more.

5. An organic light emitting diode, comprising:
an anode;
a cathode; and
at least one organic thin layer between the anode and cathode,
wherein the at least one organic thin layer includes the compound for an organic optoelectronic device as claimed in claim 1.

6. The organic light emitting diode as claimed in claim 5, wherein the compound for an organic optoelectronic device is a host material or a charge transport material.

7. The organic light emitting diode as claimed in claim 5, wherein the at least one organic thin layer includes the compound for an organic optoelectronic device singularly or as a host material along with a dopant.

8. The organic light emitting diode as claimed in claim 7, wherein the dopant is a phosphorescent dopant selected from the group of a red, green, blue, and white light emitting dopant.

9. The organic light emitting diode as claimed in claim 5, wherein the at least one organic thin layer is selected from the group of an emission layer, a hole transport layer (HTL), a hole injection layer (HIL), an electron blocking layer, an electron transport layer (ETL), an electron injection layer (EIL), a hole blocking layer, and a combination thereof.

10. The organic light emitting diode as claimed in claim 5, wherein the at least one organic thin layer further includes a compound represented by the following Chemical Formula 27. 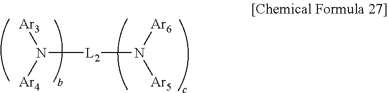
wherein, in Chemical Formula 27,
Ar₃ to Ar₆ are each independently a substituted or unsubstituted C6 to C30 aryl group,
L₂ is selected from the group of a substituted or unsubstituted C1 to C30 alkylene group, a substituted or unsubstituted C3 to C30 cycloalkylene group, a substituted or unsubstituted C6 to C30 arylene group, a substituted or unsubstituted C1 to C30 heteroarylene group, a substituted or unsubstituted C6 to C30 arylamine group, and a combination thereof, and
b and c are each independently integers of 0 to 3, provided that b and c are not simultaneously 0.

11. The organic light emitting diode as claimed in claim 10, wherein the compound represented by Chemical Formula 27 includes a compound represented by one of the following Chemical Formulae 28 to 31.

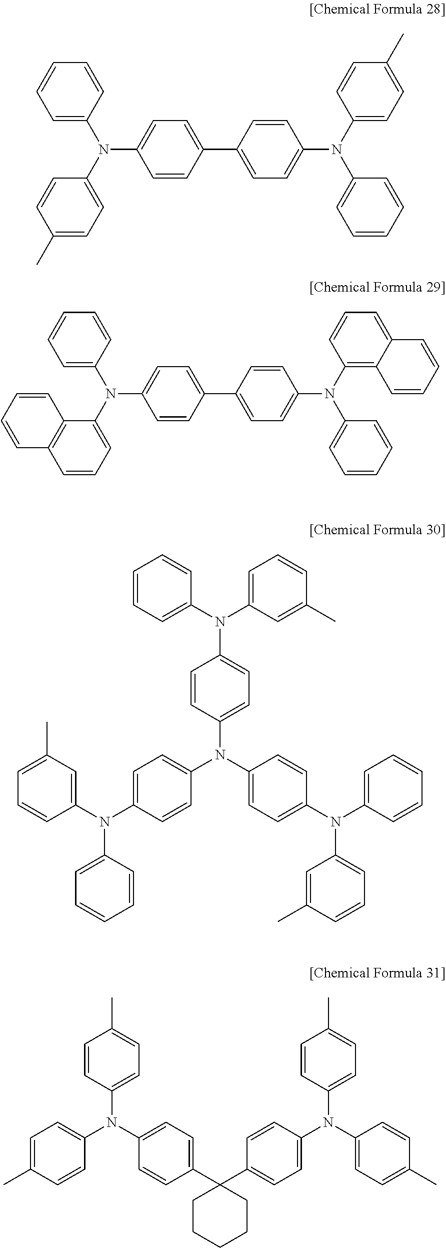

12. The organic light emitting diode as claimed in claim 10, wherein the compound for an organic optoelectronic device and the compound represented by Chemical Formula 27 are included in the at least one organic thin layer in a weight ratio of about 99:1 to about 1:99.

13. A display device comprising the organic light emitting diode as claimed in claim 5.
